(19) United States
(12) Patent Application Publication
(10) Pub. No.: US 2019/0248957 A1
(43) Pub. Date: Aug. 15, 2019

(54) BIO-REDUCIBLE POLYMERS

(71) Applicant: University of Utah Research Foundation, Salt Lake City, UT (US)

(72) Inventors: Sung Wan Kim, Salt Lake City, UT (US); Joungpyo Nam, Salt Lake City, UT (US); Soyoung Kim, Salt Lake City, UT (US)

(73) Assignee: University of Utah Research Foundation

(21) Appl. No.: 16/270,501

(22) Filed: Feb. 7, 2019

Related U.S. Application Data
(60) Provisional application No. 62/627,551, filed on Feb. 7, 2018.

(51) Int. Cl. C08G 73/02; C08G 69/42; A61K 48/00

(52) U.S. Cl. CPC C08G 73/0206; C08G 69/42; C08G 73/028; A61K 31/7088; A61K 48/0075; A61K 48/005; A61K 48/0041

(57) ABSTRACT

Bio-reducible polymers including a poly(ethyleneimine) (PEI) conjugated to poly(cystaminebis(acrylamide)-diaminohexane) (poly(CBA-DAH)) and attendant formulations and methods are disclosed.

### PRIORITY DATA

This application claims the benefit of U.S. Provisional Patent Application Ser. No. 62/627,551, filed Feb. 7, 2018, which is incorporated herein by reference.

### BACKGROUND

Gene therapy is the administration of genetic materials (e.g. plasmid deoxyribonucleic acid (pDNA), small interfering ribonucleic acid (siRNA), and messenger RNA (mRNA), etc.) into specific cells for the treatment of genetic and acquired disorders. In the early days, gene therapy only focused on inherited genetic disorders, but it has recently been applied in various disorders including different forms of cancers, vascular diseases, some autosomal dominant disorders, emphysema, retinitis pigmentosa, diabetes, hemophilia, and neurodegenerative disorders. However, there are limitations in delivering only the genetic material itself. Genetic materials have low cellular uptake efficiency due to their hydrophilic properties, large size, high anionic charge density, and susceptibility toward nuclease-mediated degradation. Therefore, carriers (commonly called vectors) have been used to solve the problem without any degradation of genetic materials.

### BRIEF DESCRIPTION OF THE DRAWINGS

Invention features and advantages will be apparent from the detailed description which follows, and are further enhanced in conjunction with the accompanying drawings, which together illustrate, by way of example, various invention embodiments.

FIG. 1 depicts a synthesis scheme and molecular structure of PCDP. i) Traut's reagent in 0.1 M sodium phosphate buffer, RT, 4 h, ii) SPDP in DMF:0.1 M sodium phosphate buffer co-solvent (1:9), RT, 4 h, iii) 50 mM phosphate buffer, RT, 12 h.

FIG. 2 is a ¹H NMR spectra of PCDP, PEI 1.8 kDa, and poly(CBA-DAH) in NMR solvent (D₂O, 3 mg/mL).

FIG. 3A is an agarose gel electrophoresis of PCDP, PEI 25 kDa, and poly(CBA-DAH) polyplexes with pDNA (gWiz-Luc). The number (0) means only naked pDNA and 0.05 to 5 (PCDP and PEI 25 kDa) and 1 to 20 (poly(CBA-DAH)) mean weight ratio based on pDNA (300 ng).

FIG. 3B is a graph of particle size and zeta-potential of PCDP/pDNA polyplexes at various weight ratios ranging from 0.1 to 20 based on pDNA (gWiz-Luc, 4 μg). Results are represented as mean±SD. (n=3).

FIG. 3C is a graph of particle size and zeta-potential of PCDP/pDNA polyplexes at a weight ratio 10 with 5 mM DTT to confirm the dissociation of polyplexes. PEI 25 kDa/pDNA polyplexes (weight ratio 1) were used as a comparison group. Results are represented as mean±SD. (n=3).

FIG. 4A is a graph of cell viability of PCDP in A549, Huh-7, and Mia PaCa-2 cells. The numbers in the bottom refer to the mean concentration of polymer ranging from 1 to 20 μg/mL. Results are represented as mean±SD. (n=3).

FIG. 4B is a graph of cell viability of PEI 25 kDa in A549, Huh-7, and Mia PaCa-2 cells. The numbers in the bottom refer to the mean concentration of polymer ranging from 1 to 20 μg/mL. Results are represented as mean±SD. (n=3).

FIG. 4C is a graph of cell viability of various polyplexes. The numbers in the bottom refer to weight ratios of PCDP/pDNA polyplexes based on pDNA (gWiz-Luc, 500 ng). Poly(CBA-DAH), PEI 25 kDa, and Lipofectamine® formed the polyplexes with pDNA at weight ratio 1:40, 1:1, and 1:2, respectively. Results are represented as mean±SD. (n=3).

FIG. 5A is a graph of cellular uptake of polyplexes in A549 cells. The YOYO-1 stained pDNA formed the polyplexes with PCDP, poly(CBA-DAH), PEI 25 kDa, and Lipofectamine® at weight ratio based on pDNA (1 μg), respectively.

FIG. 5B is a graph of cellular uptake of polyplexes in Huh-7 cells. The YOYO-1 stained pDNA formed the polyplexes with PCDP, poly(CBA-DAH), PEI 25 kDa, and Lipofectamine® at weight ratio based on pDNA (1 μg), respectively.

FIG. 5C is a graph of cellular uptake of polyplexes in Mia PaCa-2 cells. The YOYO-1 stained pDNA formed the polyplexes with PCDP, poly(CBA-DAH), PEI 25 kDa, and Lipofectamine® at weight ratio based on pDNA (1 μg), respectively.

FIG. 5D is a graph of cellular uptake % of quantification of cell internalization measured by FACs. Results are represented as mean±SD. (n=3).

FIG. 6A presents fluorescent microscopy images of GFP expression of polyplexes against A549, Huh-7, and Mia PaCa-2 cells for 48 h. The scale bar means 200 m.

FIG. 6B is a graph of the quantified and normalized GFP expression (mean). The numbers on the bottom refer to the mean weight ratios based on pDNA (gWiz-GFP, 500 ng). Results are represented as mean±SD (n=3).

FIG. 7A is a graph of luciferase expression of polymer/pDNA (gWiz-Luc, 500 ng) polyplexes in A549 cells for 48 h. Results are represented as mean±SD. (n=3). Black bars mean (−) FBS and white bars mean (+) FBS.

FIG. 7B is a graph of luciferase expression of polymer/pDNA (gWiz-Luc, 500 ng) polyplexes in Huh-7 cells for 48 h. Results are represented as mean±SD. (n=3). Black bars mean (−) FBS and white bars mean (+) FBS.

FIG. 7C is a graph of luciferase expression of polymer/pDNA (gWiz-Luc, 500 ng) polyplexes in Mia PaCa-2 cells for 48 h. Results are represented as mean±SD. (n=3). Black bars mean (−) FBS and white bars mean (+) FBS.

FIG. 8A is a graph of down regulation of VEGF expression using pshVEGF delivery into the cancer cells for 48 h by polymers. The VEGF expression were measured by human VEGF ELISA kit. Quantification of down regulated VEGF expression is shown by relative VEGF expression %. Relative VEGF expression (%)=Amount of VEGF (Treated)/Amount of VEGF (Control)×100. Results are represented as mean±SD. (n=3).

FIG. 8B is a graph of cell growth inhibition of polymers with gWiz-Luc polyplexes measured by MTT assay. Results are represented as mean±SD. (n=3).

FIG. 8C is a graph of cell growth inhibition of polymers with shVEGF polyplexes were measured by MTT assay. Results are represented as mean±SD. (n=3).

These drawings are provided to illustrate various aspects certain invention embodiments and are not intended to be limiting in scope in terms of dimensions, materials, configurations, arrangements or proportions unless otherwise limited by the claims.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this written description, the singular forms “a,” “an” and “the” include express support for plural referents unless the context clearly dictates otherwise. Thus, for example, reference to “a polymer” can include a plurality of such polymers.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, “subject” refers to a mammal that may benefit from the administration of a composition described herein. In one aspect the mammal may be a human.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients.

Compositions can take nearly any physical state, including solid and/or liquid (i.e. solution). Furthermore, the term “dosage form” can include one or more formulation(s) or composition(s) provided in a form suitable for administration to a subject.

As used herein, “effective amount” refers to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating or preventing a condition for which the active agent is known to be effective. It is understood that various biological factors may affect the ability of a substance to perform its intended task. Therefore, an “effective amount” or a “therapeutically effective amount” may be dependent in some instances on such biological factors. Additionally, in some cases an “effective amount” or a “therapeutically effective amount” may not be achieved in a single dose. Rather, in some examples, an “effective amount” or a “therapeutically effective amount” can be achieved after administering a plurality of doses over a period of time, such as in a pre-designated dosing regimen. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical and nutritional sciences as well as medicine.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 milligrams to about 80 milligrams” should also be understood to provide support for the range of “50 milligrams to 80 milligrams.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well. In some specific examples, about can mean within 10% relative to the amount stated. Thus, where this is the case, “about 100” can refer to any amount from and including 90 to 110. In some additional specific examples, about can mean within 5% relative to the amount stated. Thus, where this is the case, “about 100” can refer to any amount from and including 95 to 105.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Gene therapy is the administration of genetic materials (e.g. plasmid deoxyribonucleic acid (pDNA), small interfering ribonucleic acid (siRNA), and messenger RNA (mRNA), etc.) into specific cells for the treatment of genetic and acquired disorders. In the early days, gene therapy only focused on inherited genetic disorders, but it has recently been applied in various disorders including different forms of cancers, vascular diseases, some autosomal dominant disorders, emphysema, retinitis pigmentosa, diabetes, hemophilia, and neurodegenerative disorders. However, there are limitations in delivering only the genetic material itself. Genetic materials have low cellular uptake efficiency due to their hydrophilic properties, large size, high anionic charge density, and susceptibility toward nuclease-mediated degradation. Therefore, carriers (commonly called vectors) have been used to solve the problem without any degradation of genetic materials.

Viral vectors such as adenovirus, lentivirus, retrovirus, adeno-associated virus, and herpes simplex virus can serve as vehicles for efficient gene transfer. However, viral vectors can pose a variety of concerns including inflammation, difficulty in production, inflammatory response, immunogenicity, and carcinogenicity, for example. Non-viral vectors such as liposomes, micelles, polycationic polymer, and polyanionic polymer are alternatives that can address the concerns of viral vectors. Non-viral vectors have lower gene transfection efficiency than viral vectors, but are less toxic and immunogenic than viral vectors. In addition, non-viral vectors have some advantages such as the potential for repeated administration and ease of modification and production.

Some specific examples of non-viral vectors can include polycationic polymers. To enhance transfection efficiency, these polycationic polymers can be configured to form nano-sized polyplexes with genetic materials, have bio-reducible properties, cell penetrating properties, and endosome escape properties, to name a few. One or more of these modifications can provide a polycationic polymer with enhanced transfection efficiency, however, these modifications do not necessarily cause the polymer to be a more effective gene carrier.

The present disclosure describes a non-viral vector that has good transfection efficiency and is an effective gene carrier. Specifically, poly(ethylenimine) (PEI) can be conjugated to poly(cystaminebis(acrylamide)-diaminohexane)) (poly(CBA-DAH)) to form a non-viral vector (referred to herein as PCDP) to decrease the payload weight ratio and increase transfection efficiency of the vector. In other words, PCDP polymers can lead to formation of polyplexes with low payload weight ratios and high transfection efficiency. In some examples, PEI can be conjugated to poly(CBA-DAH) through a bio-reducible bond, such as a disulfide bond. In some examples, polyplexes of the bio-reducible polymers can have higher transfection efficiency than comparison groups including poly(CBA-DAH), PEI 25 kDa, and Lipofectamine® polyplexes.

In further detail, PEI can provide increased payload binding ability and enhanced endosome escape properties to poly(CBA-DAH). For example, PEI has many nitrogen atoms including primary, secondary, and tertiary amine groups, which can increase binding affinity with nucleic acids (e.g. pDNA, for example), buffering capacity, and provide positive charge to polyplexes. Further, these amine groups can increase cellular uptake and endosomal escape, leading to increased transfection efficiency. As such, the bio-reducible polymers described herein can have good nucleic acid binding ability in very low weight ratios.

The PEI component of the bio-reducible polymers can have a variety of molecular weights. Generally, the PEI component can have a relatively low weight average molecular weight (Mw), such as below 10 kilodaltons (kDa), for example. In some specific examples, the PEI can have an Mw of from about 500 daltons (Da) to about 5 kDa. In other examples, the PEI can have an Mw of from about 1 kDa to about 3 kDa, or about 1.5 kDa to about 2.5 kDa. In some specific examples, the PEI can have an Mw of about 1.8 kDa. Molecular weight can be measured by a variety of analytical techniques such as size exclusion chromatography, multi-angle light scattering, the like, or a combination thereof.

Poly(CBA-DAH) can minimizing cytotoxicity due to the plurality of biodegradable linkages in the poly(CBA-DAH). More specifically, poly(CBA-DAH) is composed of multiple disulfide bonds, where these disulfide bonds can be cleaved in the cytoplasm by an intracellular reducing agent such as glutathione (GSH). GSH is composed of a tri-peptide and synthesized in the cytosol from precursor amino acids. The intracellular concentration of GSH (1-10 mM) is extensively higher than extracellular levels (2 μM in plasma). These different intracellular or extracellular concentrations of GSH lead to selective intracellular release of genetic materials from the polycationic polymer, which also contains disulfide bonds.

The poly(CBA-DAH) component of the bio-reducible polymers can have a general structure according to formula I:

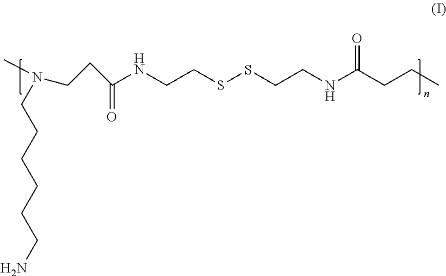

where n is an integer from 1 to 12. In some additional examples, n can be an integer from 2 to 10, or from 4 to 8. Further, in some examples, the poly(CBA-DAH) component can include one or more substitutions, as desirable.

The PEI component can be conjugated to the poly(CBA-DAH) component via a variety of linkages. In some examples, the PEI component and the poly(CBA-DAH) component can be conjugated together via a non-biodegradable linkage. In other examples, the PEI component and the poly(CBA-DAH) component can be conjugated together via a bio-reducible or biodegradable linkage. For example, in some cases, the bio-reducible polymers can include a bio-reducible linkage including a disulfide bond, an amide bond, an ester bond, an ether bond, the like, or a combination thereof that can facilitate bio-reduction or biodegradation of the conjugation linkage. In some specific examples, the bio-reducible linkage can include a disulfide bond.

The PEI component can be conjugated to the poly(CBA-DAH) component in a variety of ratios. For example, in some cases PEI can have a conjugation ratio to poly(CBA-DAH) of from 1 to 8. In other examples, PEI can have a conjugation ratio to poly(CBA-DAH) of from 2 to 6, or 3 to 5. In some specific examples, PEI can have a conjugation ratio to poly(CBA-DAH) of 4 (i.e. 4 PEI units per 1 poly(CBA-DAH) unit). The PEI units can be conjugated to poly(CBA-DAH) in a variety of configurations or patterns. For example, in some cases, a PEI unit can be conjugated to each poly(CBA-DAH) repeat unit. In other examples, PEI units can be conjugated to alternating poly(CBA-DAH) repeat units. In other examples, PEI units can be conjugated to poly(CBA-DAH) repeat units in a random pattern. In some further examples, PEI units can be conjugated to poly(CBA-DAH) repeat units in blocks, such as contiguous blocks, alternating blocks, random blocks, or the like.

In some specific examples, the bio-reducible polymers can have a general structure according to formula II:

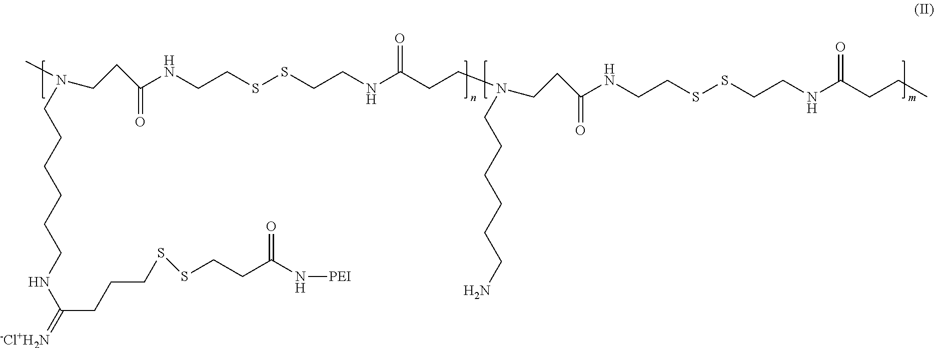

where n is an integer from 1 to 8 and m is an integer from 1 to 8. In some additional examples, n can be an integer from 2 to 6. In some further examples, m can be an integer from 2 to 6. It is further noted, that the bio-reducible polymer having the general structure of formula (II) can also include one or more substitutions, as desired.

As noted above, the PEI component includes a number of amine groups, which can increase the buffer capacity of the bio-reducible polymers as compared to poly(CBA-DAH) alone. For example, in some cases the bio-reducible polymers can have a buffer capacity that is at least 5% greater than poly(CBA-DAH) alone. In other examples, the bio-reducible polymers can have a buffer capacity that is at least 10% greater than poly(CBA-DAH) alone. In some other examples, the bio-reducible polymers can have a buffer capacity that is at least 15% greater than poly(CBA-DAH) alone.

As described above, PCDP polymers can lead to formation of polyplexes with low payload weight ratios and high transfection efficiency. Thus, the present disclosure also describes therapeutic agents (e.g. polyplexes, for example) that can include a bio-reducible polymer as disclosed herein. For example, the therapeutic agents can include a therapeutic nucleic acid carried by a bio-reducible polymer as described herein.

In further detail, variety of therapeutic nucleic acids can be included in the therapeutic agent. A few non-limiting examples can include a plasmid deoxyribonucleic acid (pDNA), an anti-sense oligonucleotides (ASO), an aptamer, a ribozyme, a small interfering ribonucleic acid (siRNA), a microRNA (miRNA), a messenger RNA (mRNA), the like, or a combination thereof. In some specific examples, the therapeutic nucleic acid can include a pDNA.

The bio-reducible polymer and the therapeutic nucleic acid can be present at a variety of weight ratios in the therapeutic agents. In some examples, the bio-reducible polymer and the therapeutic nucleic acid can be present at a weight ratio of from 0.5 to 20 polymer to therapeutic nucleic acid. In some other examples, the bio-reducible polymer and the therapeutic nucleic acid can be present at a weight ratio of from 1 to 15 polymer to therapeutic nucleic acid. In still other examples, the bio-reducible polymer and the therapeutic nucleic acid can be present at a weight ratio of from 8 to 17 polymer to therapeutic nucleic acid.

The therapeutic agent can have a variety of particle sizes. In some examples, the therapeutic agent can have a particle size of from about 90 nm to about 150 nm. In other examples, the therapeutic agent can have a particle size of from about 100 nm to about 140 nm. In still other examples, the therapeutic agent can have a particle size of from about 110 nm to about 130 nm.

Additionally, the therapeutic agent can have various zeta potentials. In some examples, the therapeutic agent can have a zeta potential of from about 20 mV to about 40 mV. In some other examples, the therapeutic agent can have a zeta potential of from about 25 mV to about 35 mV.

The present disclosure also describes therapeutic compositions that can include a therapeutic agent as described herein and a pharmaceutically acceptable carrier. The therapeutic composition can include a variety of pharmaceutically acceptable carriers depending on the delivery modality for therapeutic agent.

In some examples, the pharmaceutically acceptable carrier can be formulated to provide a therapeutic composition for administration via injection, such as intramuscular injection, intravenous injection, subcutaneous injection, intradermal injection, intrathecal injection, intraocular injection, or the like. In such examples, the pharmaceutically acceptable carrier can include a variety of components, such as water, a solubilizing or dispersing agent, a tonicity agent, a pH adjuster or buffering agent, a preservative, a chelating agent, a bulking agent, the like, or a combination thereof.

In some examples, an injectable therapeutic composition can include a solubilizing or dispersing agent. Non-limiting examples of solubilizing or dispersing agents can include polyoxyethylene sorbitan monooleates, lecithin, polyoxyethylene polyoxypropylene co-polymers, propylene glycol, glycerin, ethanol, polyethylene glycols, sorbitol, dimethylacetamide, polyethoxylated castor oils, n-lactamide, cyclodextrins, caboxymethyl cellulose, acacia, gelatin, methyl cellulose, polyvinyl pyrrolidone, the like, or combinations thereof.

In some examples, an injectable therapeutic composition can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus, the like, or combinations thereof. The tonicity agent can be used to provide an appropriate tonicity of the therapeutic composition. In one aspect, the tonicity of the therapeutic composition can be from about 250 to about 350 milliosmoles/liter (mOsm/L). In another aspect, the tonicity of the therapeutic composition can be from about 277 to about 310 mOsm/L.

In some examples, an injectable therapeutic composition can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8.

In some examples, an injectable therapeutic composition can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In some examples, an injectable therapeutic composition can include a chelating agent. Non-limiting examples of chelating agents can include ethylenediaminetetra acetic acid, calcium, calcium disodium, versetamide, calteridol, diethylenetriaminepenta acetic acid, the like, or combinations thereof.

In some examples, an injectable therapeutic composition can include a bulking agent. Non-limiting examples of bulking agents can include sucrose, lactose, trehalose, mannitol, sorbitol, glucose, rafinose, glycine, histidine, polyvinyl pyrrolidone, the like, or combinations thereof.

In yet other examples, the pharmaceutically acceptable carrier can be formulated to provide a therapeutic composition for enteral administration, such as via solid oral dosage forms or liquid oral dosage forms. In the case of solid oral dosage forms, the pharmaceutically acceptable carrier can include a variety of components suitable for forming a capsule, tablet, or the like. In the case of a liquid dosage form, the pharmaceutically acceptable carrier can include a variety of components suitable for forming a dispersion, a suspension, a syrup, an elixir, or the like.

In some specific examples, the therapeutic composition can be formulated as a tablet. In such examples, the therapeutic composition can typically include a binder.

Non-limiting examples of binders can include lactose, calcium phosphate, sucrose, corn starch, microcrystalline cellulose, gelatin, polyethylene glycol (PEG), polyvinyl pyrrolidone (PVP), hydroxypropyl cellulose, hydroxyethylcellulose, carboxymethyl cellulose (CMC), the like, or combinations thereof.

Where the therapeutic composition is formulated as a tablet, in some examples the therapeutic composition can also include a disintegrant. Non-limiting examples of disintegrants can include crosslinked PVP, crosslinked CMC, modified starch, sodium starch glycolate, the like, or combinations thereof.

In some examples the tablet can also include a filler. Non-limiting examples of fillers can include lactose, dicalcium phosphate, sucrose, microcrystalline cellulose, the like, or combinations thereof.

In some further examples, the tablet can include a coating. Such coatings can be formed with a variety of materials, such as hydroxypropyl methylcellulose (HPMC), shellac, zein, various polysaccharides, various enterics, the like, or combinations thereof.

In some examples, the tablet can include a variety of other ingredients, such as anti-adherents (e.g. magnesium stearate, for example), colorants, glidants (e.g. fumed silica, talc, magnesium carbonate, for example), lubricants (e.g. talc, silica, magnesium stearate, stearic acid, for example) preservatives, desiccants, and/or other suitable tablet excipients, as desired.

In some other examples, the therapeutic composition can be formulated as a capsule. In such examples, the capsule itself can typically include gelatin, hypromellose, HPMC, CMC, the like, or combinations thereof. A variety of excipients can also be included within the capsule, such as binders, disintegrants, fillers, glidants, preservatives, coatings, the like, or combinations thereof, such as those listed above with respect to tablets, for example, or other suitable variations.

In some examples, the therapeutic composition can be formulated as a liquid oral dosage form. A liquid oral dosage form can include a variety of excipients, such as a liquid vehicle, a solubilizing agent, a thickener or dispersant, a preservative, a tonicity agent, a pH adjuster or buffering agent, a sweetener, the like, or a combination thereof.

Non-limiting examples of liquid vehicles can include water, ethanol, glycerol, propylene glycol, the like, or combinations thereof. Non-limiting examples of solubilizing agents can include banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof. Non-limiting examples of thickeners or dispersants can include sodium alginate, methylcellulose, hydroxyethylcellulose, hydroxypropylcellulose, HPMC, CMC, microcrystalline cellulose, tragacanth, xanthangum, bentonite, carrageenan, guar gum, colloidal silicon dioxide, the like, or combinations thereof. The preservative, tonicity agent, pH adjuster or buffering agent can typically be any of those described above with respect to the injectable formulations or other suitable preservative, tonicity agent, pH adjuster or buffering agent.

Sweeteners can include natural and/or artificial sweeteners, such as sucrose, glucose, fructose, stevia, erythritol, xylitol, aspartame, sucralose, neotame, acesulfame potassium, saccharin, advantame, sorbitol, the like, or combinations thereof, for example.

In yet other examples, the pharmaceutically acceptable carrier can be formulated to provide a therapeutic composition for topical, transdermal, or transmucosal administration, such as to the skin, to the eye, to the vaginal cavity, to the rectum, to the nasal cavity, the like, or a combination thereof. Further, the topical formulations can be formulated for local and/or systemic delivery of one or more components of the therapeutic composition.

Where the therapeutic composition is formulated for topical, transdermal, or transmucosal administration, the pharmaceutically acceptable carrier can include a variety of components suitable for forming a suspension, dispersion, lotion, cream, ointment, gel, foam, patch, powder, paste, sponge, the like, or a combination thereof.

Non-limiting examples can include a solubilizer, an emulsifier, a dispersant, a thickener, an emollient, a pH adjuster, a tonicity agent, a preservative, an adhesive, a penetration enhancer, the like, or a combination thereof. Non-limiting examples of solubilizers and/or emulsifiers can include water, ethanol, propylene glycol, ethylene glycol, glycerin, polyethylene glycol, banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof. In some examples, the solubilizer can also include a hydrocarbon or fatty substance, such as petrolatum, microcrystalline wax, paraffin wax, mineral oil, ceresi, coconut oil, bees wax, olive oil, lanolin, peanut oil, spermaceti wax, sesame oil, almond oil, hydrogenated castor oils, cotton seed oil, soybean oil, corn oil, hydrogenated sulfated castor oils, cetyl alcohol, stearyl alcohol, oleyl alcohol, lauryl alcohol, myristyl alcohol, stearic acid, oleic acid, palmitic acid, lauraic acid, ethyl oleate, isopropyl myristicate, the like, or combinations thereof. In some examples, the solubilizer can include a silicon, such as polydimethylsiloxanes, methicones, dimethylpropylsiloxanes, methyl phenyl polysiloxanes, steryl esters of dimethyl polysiloxanes, ethoxylated dimethicones, ethoxylated methicones, the like, or combinations thereof.

In some additional examples, the therapeutic composition can include a dispersant and/or thickening agent, such as polyacrylic acids (e.g. Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof.

In some examples, the therapeutic composition can include an emollient, such as aloe vera, lanolin, urea, petrolatum, shea butter, cocoa butter, mineral oil, paraffin, beeswax, squalene, jojoba oil, coconut oil, sesame oil, almond oil, cetyl alcohol, stearyl alcohol, olive oil, oleic acid, triethylhexanoin, glycerol, sorbitol, propylene glycol, cyclomethicone, dimethicone, the like, or combinations thereof.

In some examples, the therapeutic composition can include an adhesive, such as acrylic adhesives, polyisobutylene adhesives, silicon adhesives, hydrogel adhesives, the like, or combinations thereof.

In some examples, the therapeutic composition can include a penetration enhancer, such as ethanol, propylene glycol, oleic acid and other fatty acids, azone, terpenes, terpenoids, bile acids, isopropyl myristate and other fatty esters, dimethyl sulphoxides, N-methyl-2-pyrrolidone and other pyrrolidones, the like, or combinations thereof.

The pH adjusters, tonicity agents, and preservatives in the topical, transdermal, or transmucosal therapeutic composition can generally include those pH adjusters and buffering agents, tonicity agents, and preservative agents listed above, or any other suitable pH adjusters, buffering agent, tonicity agent, or preservative for a particular formulation and/or use thereof. In some examples, the therapeutic composition can also include fumed silica, mica, talc, titanium dioxide, kaolin, aluminum glycinate, ethylenediaminetetraacetic acid, fragrances, colorants, other components as described above, the like, or combinations thereof.

Depending on the mode of delivery, the therapeutic agent can be present in the therapeutic composition in a variety of amounts. In some examples, the therapeutic agent can be present in the therapeutic composition in an amount of from about 0.0001 wt % to about 10 wt % based on a total weight of the therapeutic composition. In some further examples, the therapeutic agent can be present in the therapeutic composition in an amount from about 0.0001 wt % to about 0.01 wt %, from about 0.01 wt % to about 0.1 wt %, from about 0.1 wt % to about 1 wt %, or from about 1 wt % to about 10 wt % based on a total weight of the therapeutic composition. In other examples, the therapeutic agent can be present in the therapeutic composition in an amount from about 0.0005 wt % to about 0.05 wt %, from about 0.05 wt % to about 0.5 wt %, or from about 0.5 wt % to about 5 wt % based on a total weight of the therapeutic composition.

The present disclosure also describes methods of delivering a therapeutic nucleic acid to a target cell. The methods can include administering a therapeutic agent as described herein to the target cell. This can be done in a variety of ways. In some examples, the therapeutic agent can be administered in vivo (e.g. directly to a subject) in a therapeutically effective amount, a therapeutic regimen, the like, or a combination thereof. In vivo administration can be performed in a variety of ways, such as via oral administration, transmucosal administration, injection, topical administration, transdermal administration, the like, or a combination thereof.

Thus, the therapeutic agent can be administered at a variety of suitable doses and frequencies depending on the therapeutic agent, the condition being treated, etc. For example, in some cases, administration of the therapeutic agent can be performed once per day, twice per day, three times per day, four times per day, or more. In some further examples, daily administration (e.g. once, twice, three times, four times per day, etc.) can be performed once, every day, every two days, every three to five days, once per week, once every two weeks, once every three weeks, once per month, once every three months, once every six months, or the like during a treatment period. Treatment periods can be determined by medical personnel and can generally range from a period of one day to perpetual. In some examples, the treatment period can be from about 4 weeks to about 8 months, or from about 6 months to about 18 months, or from about 12 months to about 36 months or about 48 months.

In other examples, the target cell can be removed from a subject and the therapeutic agent can be administered in vitro to the target cell in a therapeutically effective amount. The treated target cell can then be re-introduced to the subject. In some additional examples, administration of the therapeutic agent to the target cell can result in modified gene expression of the target cell. In some examples, modified gene expression can result from gene therapy. In other examples, modified gene expression can result from inhibition of gene expression.

As described herein, the therapeutic agent can have a relatively high transfection efficiency. As such, in some examples, administration of the therapeutic agent can yield or result in a higher transfection efficiency than administration of an equivalent therapeutic nucleic acid carried by poly(CBA-DAH) alone.

A number of additional features of the bio-reducible polymers, therapeutic agents, therapeutic compositions, and associated methods will also be apparent from the following examples.

### EXAMPLES

The following materials were used in one or more of the following examples: gWiz-Luc and gWiz-GFP were amplified in E. coli DH5a and isolated by NucleoBond® Xtra Maxi Plus EF kit (MACHEREY-NAGEL GmbH & Co. KG, Dfiren, Germany). The luciferase assay kit, agarose, and 5× reporter lysis buffer were purchased from Promega (Madison, Wis.). pshVEGF was designed and conducted according to our previous work²². tert-Butyl-N-(6-aminohexyl) carbamate (N-Boc-1,6-diaminohexane, N-Boc-DAH), 3-[4,5-dimethylthiazol-2-yl]-2,5-diphenyltetrazolium bromide (MTT), 1,4-dithiothreitol (DTT), trifluoroacetic acid (TFA), piperidine, N,N-diisopropylethylamine (DIPEA), triisopropylsilane (TIS), dimethylformamide (DMF), dimethyl sulfoxide (DMSO), methanol (MeOH), and trypan blue solution (0.4%) were purchased from Sigma-Aldrich (St. Louis, Mo.). Fetal bovine serum (FBS) was purchased from Seradigm (Radnor, Pa.). Branched poly(ethylenimine) (bPEI, Mw=1.8 kDa and 25 kDa), N,N′-Cystaminebisacrylamide (CBA) was purchased from PolySciences, Inc. (Warrington, Pa.). Spectrapor dialysis membrane was purchased from Spectrum Laboratories, Inc. (Rancho Dominguez, Calif.). Dulbecco's modified Eagle's medium (DMEM), human VEGF ELISA kit, YOYO-1 iodide (1 mM solution in DMSO), Lipofectamine® 2000 (DNA transfection reagent, 1 mg/mL), Opti-MEM® medium, trypsin-like enzyme (TrypLE™ Express), Dulbecco's phosphate buffered saline (DPBS), and propidium iodide (PI, 1 mg/mL in H₂O) were purchased from Invitrogen (Carlsbad, Calif.). Ellman's reagent, Traut's reagent, succinimidyl 3-(2-pyridyldithio) propionate) (SPDP) cross linker and BCA assay kit were purchased from pierce (Rockford, Ill.).

Example 1—Synthesis of the PEI conjugated Poly(CBA-DAH) (PCDP) To increase the transfection efficiency and decrease the weight ratio when the polyplexes formed with pDNA, a novel bio-reducible polymer (PCDP) with poly(CBA-DAH) and PEI 1.8 kDa was designed. PEI can introduce high binding ability with pDNA as well as enhanced endosome escapeability of pDNA from the polyplexes, leading to decreased weight ratio and increased transfection efficiency.

Specifically, poly(CBA-DAH), the backbone of PCDP (poly(disulfide amine)), was synthesized. To introduce the sulfhydryl group, purified poly(CBA-DAH) was dissolved in a non-amine buffer (0.1 M sodium phosphate buffer), then 4.4 equiv. of Traut's reagent was added to the polymer in solution for 4 h at room temperature. After 4 h, the reacted solution was dialyzed against ultrapure water using a dialysis membrane (dialysis membrane (MWCO=3,000)) to remove unreacted Traut's reagent. Then, the product (poly(CBA-DAH)-SH) was filtered and lyophilized. The produced sulfydryl groups were measured using Ellman's reagent.

To react between the activated NHS ester of SPDP and amine of PEI, PEI (Mw; 1.8 kDa) was dissolved in 0.1 M phosphate buffer. 1.2 equiv. of SPDP dissolved in DMF and then added to PEI solution (final solvent ratio; 1:9 (DMF:0.1 M phosphate buffer)) for 4 h at room temperature. The product (PEI-SPDP) was dialyzed against ultrapure water using a dialysis membrane (dialysis membrane (MWCO=1,000)) and lyophilized.

Eight equiv. of PEI-SPDP was added to a prepared poly(CBA-DAH)-SH solution in 50 mM phosphate buffered saline and the mixture was stirred for 12 h at room temperature. The released pyridine-2-thione (leaving group) was monitored by UV spectroscopy at 343 nm to confirm the reaction. The final product (PCDP) was dialyzed against ultrapure water using a dialysis membrane (dialysis membrane (MWCO=10,000)) and was filtered, followed by lyophilization. The synthesis of PCDP was estimated by measuring ¹H NMR (Bruker, 400 MHz, D₂O).

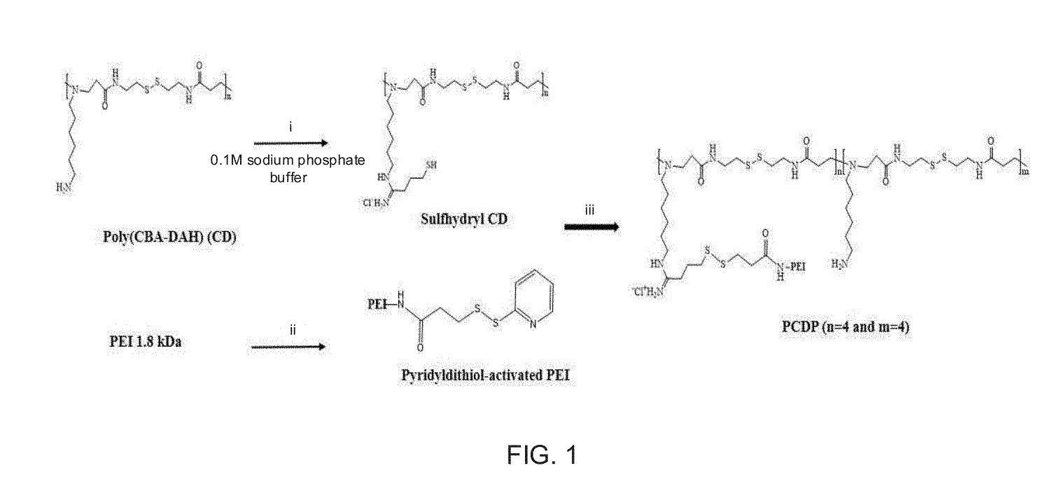

In further detail, FIG. 1 shows the synthetic route of PCDP. To introduce the disulfide bond between poly(CBA-DAH) and PEI 1.8 kDa, Traut's reagent and SPDP were used. The conjugation ratio of sulfydryl group was measured by Ellman's reagent and each reaction step was monitored by Thin-Layer Chromatography (TLC). In the first attempt, PCDPs were designed with different conjugation ratios of PEI such as 1, 4, and 8, to compare the effect of the conjugation ratio. However, in the case of conjugation ratio of 1, there was not a significant difference when compared to poly(CBA-DAH), and in the case of conjugation ratio of 8, it aggregated during the final synthesis step (data not shown). Therefore, PCDP with conjugation ratio of 4 was used for experiments to confirm its potential as a gene delivery carrier. The synthesis and conjugation ratio of PCPD were confirmed by ¹H NMR.

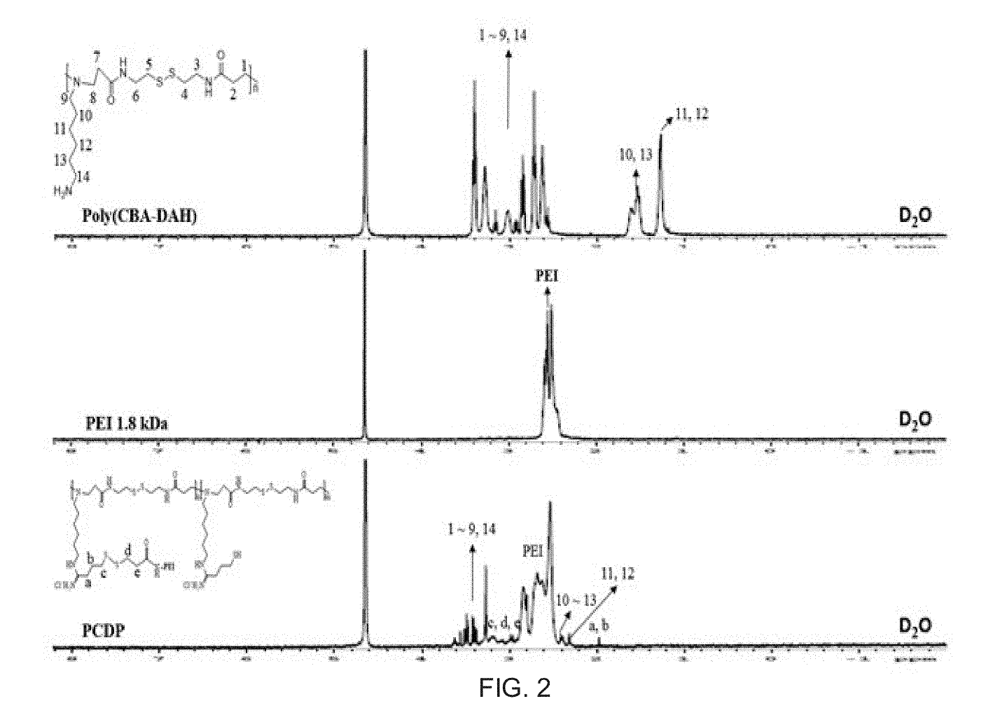

As shown in FIG. 2, the peak assignments of poly(CBA-DAH), PEI 1.8 kDa, and PCDP were classified in the data. In the NMR data of PCDP, the proton peaks of poly(CBA-DAH) and PEI were shifted downfield due to steric hindrance caused by the conjugation between poly(CBA-DAH) and PEI. In addition, the conjugation ratio of PEI to the poly(CBA-DAH) was calculated by the ratio of the integration of the proton spectrum peaks in the poly(CBA-DAH) (—NCH₂CH₂CH₂CH₂CH₂C NH₂, 4H) and CH₂ of PEI (44H). The calculated conjugation ratio by ¹H NMR analysis is shown in Table 1.

TABLE 1
Characterization of PCDP

| | Conjugation ratio (%) | | Buffer |
|---|---|---|---|
| | expected | calc^{a} | Capacity^{b} (%) |
| Poly (CBA-DAH) | — | — | 55 |
| PCDP | 50 | 50 | 67 |

^{a}Calculated PEI conjugation ratio of PCDP by ¹H NMR analysis.
^{b}Calculated by base-acid profiles.

The ¹H NMR results showed that the PCDP were successfully synthesized. The occurrence of PCDP spectrum peaks were classified as follows:

PCDP (¹H NMR, D₂O): poly(CBA-DAH) (NCH₂CH₂CH₂CH₂CH₂CH₂NH₂)=1.16 ppm (shifted to 2.25 ppm), poly(CBA-DAH) (NCH₂CH₂CH₂CH₂CH₂CH₂NH₂)=1.48 ppm (shifted to 2.4 ppm), poly(CBA-DAH) (NCH₂CH₂CONH), (CH₂SSCH₂), (NCH₂CH₂CH₂CH₂CH₂CH₂NH₂), (NCH₂CH₂CONH, NCH₂CH₂CH₂CH₂CH₂CH₂NH₂), and (NCH₂CH₂CONHCH₂)=2.57-3.43 ppm (shifted to 3.15-3.62 ppm), PEI (NCH₂CH₂N)=2.39 2.61 ppm (shifted to 2.41-2.85), cross-linker (N═CCH₂CH₂CH₂S)=1.98 ppm, cross-linker (N═CCH₂CH₂CH₂S) and (SCH₂CH₂C═O)=2.94-3.21 ppm.

### Example 2 Buffering Capacity of PCDP

The buffering capacity of PCDP was measured by acid-base titration. 10 mg of each poly(CBA-DAH) and PCDP were dissolved in 10 mL of H₂O (1 mg/mL). The solutions were adjusted to pH=10 by 0.1 M NaOH and then titrated to pH=3 with 0.01 M HCl.

PEI has many nitrogen atoms including primary, secondary, and tertiary amine groups. These amine groups can enhance the buffering capacity and cause the subsequent endosomal or lysosomal rupture and escape into the cytoplasm via a “proton sponge effect.” Therefore, PEI can provide enhanced buffering capacity to the PCDP, leading to increased endosomal escape into cytoplasm, as described above.

The buffering capacity of PCDP in the pH range of 10 to 3 was measured by acid-base titration assay and was calculated from the acid-base titration curve (data not shown) in the endosomal pH range (from pH 7.4 to 5.1) according to the following equation: Buffering capacity (%)=(ΔV HCl (volume of HCl solution)×0.01 M)/N mol (total moles of protonable amine group)×100%.

PCDP showed increased buffering capacity more than poly(CBA-DAH), as shown in Table 1. Because more amine groups were introduced to poly(CBA-DAH), where the additional amine groups are attributed to the conjugation of PEI. This result supported that the enhanced buffering capacity may help endosomal escape of the polyplexes.

### Example 3—Characterization of PCDP/pDNA Polyplexes

PCDP was dissolved in HEPES buffer (10 mM HEPES, 1 mM NaCl, pH 7.4) at 10 mg/mL and diluted (1 mg/mL or 5 mg/mL) to form polyplexes, then mixed at a certain weight ratio ranging from 0.05 to 5 based on 300 ng of pDNA (gWiz-Luc). The mixtures were then incubated at room temperature for 30 min before use. Gel retardation assay was used to evaluate the pDNA condensation ability of PCDP. After 30 min, the incubated mixtures were loaded on a 0.8% agarose gel with PI-included loading dye (5% in 10× loading dye, v/v), followed by electrophoresis in TAE buffer (10 mM tris/HCl, 1% v/v acetic acid, 1 mM EDTA) at 100V for 40 min. Naked pDNA was used as a control. The migration of PI-stained pDNA in the agrarose gel was visualized by an UV illuminator (Gel Documentation System, Bio-Rad, Hercules, Calif.). In addition, PEI 25 kDa and poly(CBA-DAH) were used as comparison groups with same (PEI 25 kDa, w/w, 0.05 to 5) or different (poly(CBA-DAH), w/w, 1 to 20) conditions.

The particle size and zeta-potential of PCDP/pDNA polyplexes were measured by dynamic light scattering (DLS) and laser Doppler velocimetry (LDV), respectively by a Nano ZS (ZEN3600, Malvern Instruments, UK). The polyplexes were prepared in HEPES buffer (10 mM HEPES, 1 mM NaCl, pH 7.4) at various weight ratios ranging from 0.05 to 5 based on 4 μg of pDNA (gWiz-Luc). After incubation time (30 min), the polyplexes solutions were diluted using distilled water to 600 μL before measurement. In addition, to confirm the dissociation of polyplexes by a reducing agent such as DTT, the particle size and zeta-potential of PCMD/pDNA polyplexes (weight ratio 10) were measured with or without 5 mM DTT. PEI was used as a comparison group (weight ratio 1).

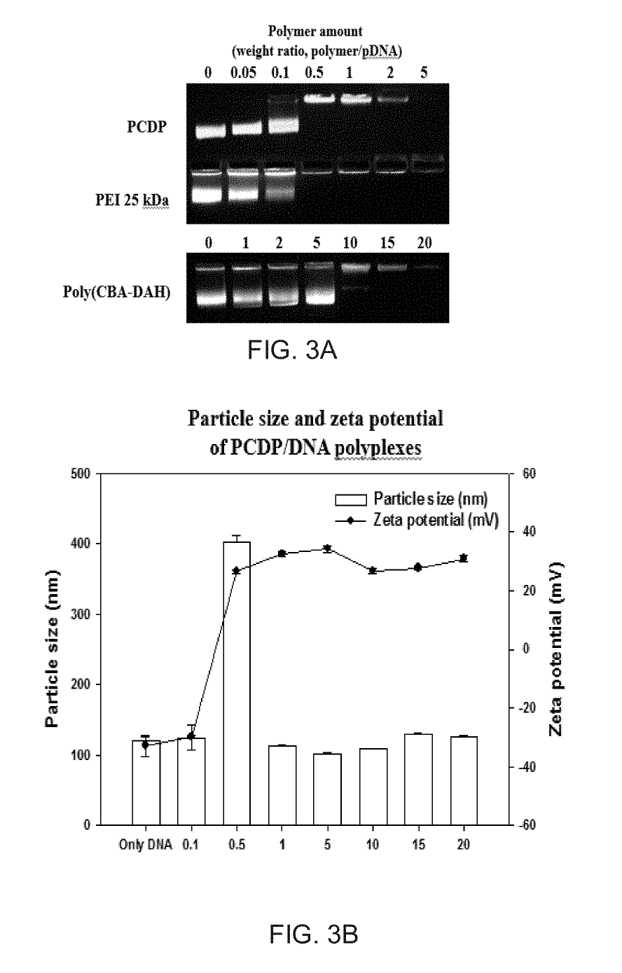

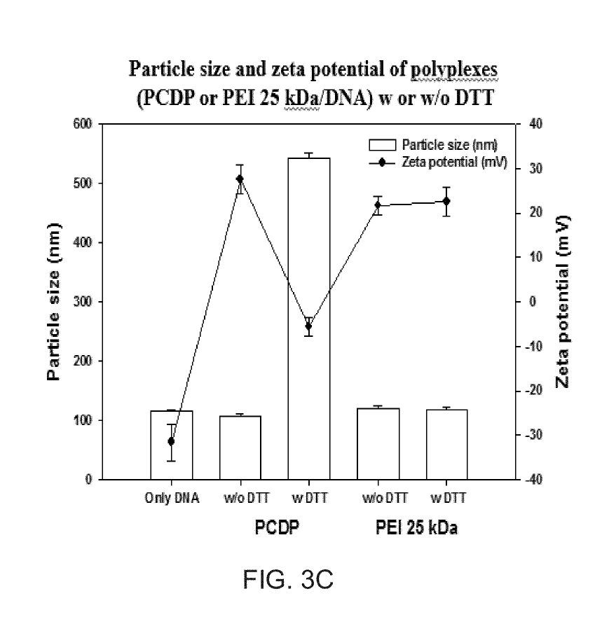

Naked pDNA are prevented from entering the cell membrane due to their hydrophilicity and negative charge. The polycationic polymer, with its ability to form polyplexes with pDNA, provides a way to overcome this problem. The binding ability between pDNA and PCPD was evaluated by retardation of the pDNA (gWiz-Luc) in gel electrophoresis, displayed in FIG. 3A. PEI 25 kDa and poly(CBA-DAH) were used as comparison groups. The bend of pDNA, which formed the polyplexes with poly(CBA-DAH), displayed pDNA migration at weight ratio of 10 or 15. On the other hand, PCDP could bind pDNA thoroughly at a very low weight ratio of 0.5 and even displayed characteristics similar to PEI 25 kDa. This gel retardation assay data indicated that PCDP had better DNA binding capacity than poly(CBA-DAH) due to increased amine groups from conjugation with PEI. This enhanced binding capacity can decrease the amount of polymer needed for successful delivery and easy application.

The positive surface charge and appropriate size (50 to several hundred nanometers) of polyplexes are important factors that affect the cellular uptake of the polyplexes. PCDP/pDNA polyplexes were formed by electrostatic interaction between PCDP and pDNA (gWiz-Luc) at various weight ratios. The particle size and zeta potential results of PCDP/pDNA polyplexes are displayed in FIG. 3B. The particle sizes and zeta potentials of PCDP/pDNA were about 102-128 nm and 27-34 mV at weight ratios of 0.5 to 20, respectively. This decreased particle size of PCDP/pDNA polyplexes is considered to be due to the formation of tighter polyplexes compared to poly(CBA-DAH)/pDNA polyplexes because of the increased presence of amine groups in PCDP. The negative value (−30 mV) of PCDP was measured at a weight ratio of 0.1, suggesting that the polyplexes are not formed at that ratio. At a weight ratio of 0.5, the zeta potential was dramatically increased to 27 mV. This result indicated that polyplexes can form from weight ratios above 0.5, which is also consistent with the gel retardation assay. However, the particle size was about 400 nm due to the unstable polyplex formation and big cluster formation by self-aggregation. The stable polyplexes of PCDP were formed at weight ratios of 1 and above.

The polycationic polymer which contains disulfide bonds can easily release pDNA after cleaved disulfide bond by reducing reagent. To confirm the change in polyplexes through the cleaved disulfide bond of PCDP, DTT (5 mM) was used as an intracellular reducing agent like GSH. As shown in FIG. 3C, the particle size and zeta potential of PEI 25 kDa/pDNA polyplexes were not influenced by 5 mM DTT. Meanwhile, the particle size of PCPD/pDNA polyplexes with 5 mM DTT dramatically increased to 542 nm and zeta potential was negative charged (−6 mV). The cleavage of the disulfide bond of bio-reducible polymer by reducing agent increased the particle size due to the decreased binding ability.

### Example 4—Cell Culture and Cytotoxicity Assay

Human lung adenocarcinoma epithelial (A549), human hepatocellular carcinoma (Huh-7), and human pancreatic carcinoma (Mia PaCa-2) cell lines were selected and obtained from American Type Culture Collection (ATCC) (Manassas, Va.). The cells were cultured in high glucose DMEM supplemented with heat-inactivated FBS (10%, v/v), without antibiotic agent at 37° C. in a humidified atmosphere containing CO₂ (5%, v/v).

The cytotoxicity of PCDP and PCDP/pDNA polyplexes were evaluated in cultured cells assessing cell killing using the MTT assay. When the A549, Huh-7, and Mia PaCa-2 cells reached 70-80%, the cells were harvested by trypsinization and centrifugation, then seeded in a 24-well plate at a density of 1×10⁴ cells/well in full DMEM medium. After 24 h, the culture medium was replaced by serum-free DMEM, then the cells were treated with polymers (PCDP and PEI 25 kDa) or polyplexes (PCDP, PEI 25 kDa, poly(CBA-DAH), and Lipofectamine® polyplexes with pDNA (gWiz-Luc)). The concentration range of the treated polymers (PCDP and PEI 25 kDa) was 1-20 g/mL. In addition, PCDP/pDNA polyplexes were prepared at various weight ratios ranging from 1 to 20, based on pDNA (gWiz-Luc, 500 ng). Poly(CBA-DAH), PEI 25 kDa, and Lipofectamine® were used as comparison groups and formed polyplexes with pDNA at 1:40, 1:1, and 1:2 weight ratio, respectively. After 4 h, the medium was replaced by serum-containing DMEM (10% FBS, without antibiotic agent) and the cells were further incubated for 48 h. 30 μL stock solution of MTT (2 mg/mL in PBS) was added to each well, and the plates were incubated for another 2 h. The medium and unreacted MTT were removed by aspiration, then 300 μL of DMSO was added to each well to dissolve the formazan crystals formed in live cells. The absorbance of each well was measured at 570 nm using a microplate reader (Model 680, Bio-Rad Laboratory, Hercules, Calif.), and the relative cell viability (%) calculated as: Cell viability (%)=((OD sample)−(OD blank))/((OD control)−(OD blank))×100%.

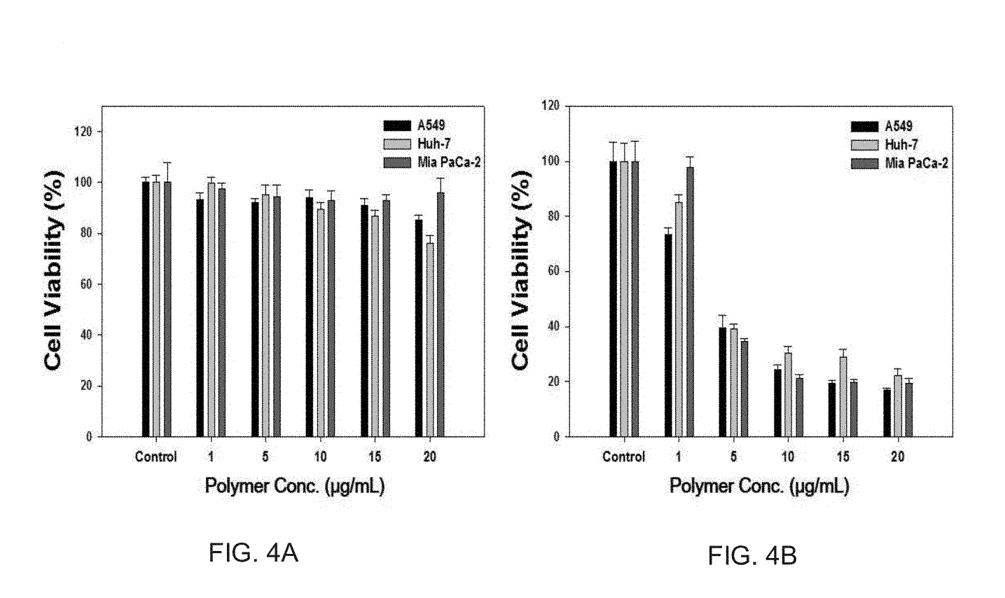

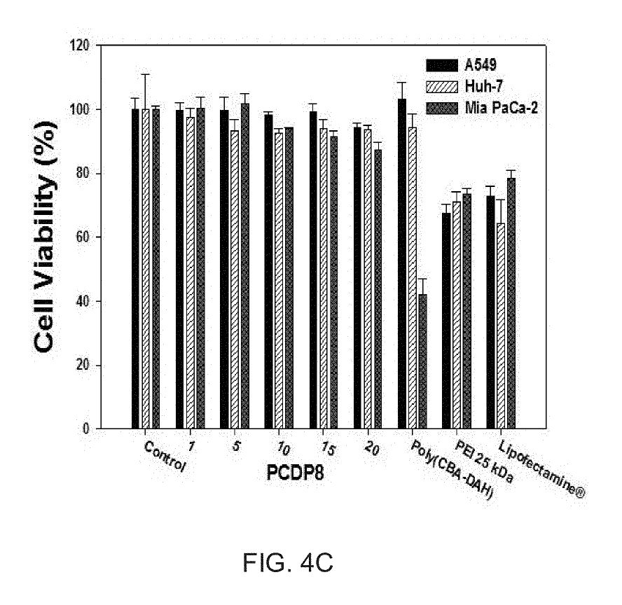

To confirm the cytotoxicity of polymer and polyplexes in A549, Huh-7, and Mia PaCa-2 cells, the MTT assay was performed. When the concentration of the treated PCPD was increased from 1 to 20 μg/mL, the cell viability was slightly decreased, as shown in FIG. 4A. However, the used maximum amount of PCDP to form the polyplexes with pDNA was 10 μg/mL and the cell viability was above 90% except in Huh-7 cells. As shown in FIG. 4B, the cell viability of PEI 25 kDa as a comparison group decreased significantly with increasing concentration up to 20 μg/mL. Generally, despite the increased transfection efficiency, PEI conjugated polycationic derivatives always had a problem with toxicity even PEI 600 Da or PEI 1.8 kDa due to the increased molecular weight from conjugation or polymerization. PCDP had no considerable cytotoxicity because PCDP consists of disulfide and amide bonds that can be degraded into non-toxic small molecules in cells. As shown in FIG. 4C, the PCDP/pDNA polyplexes were also observed to have no considerable cytotoxicity at all polyplexes formation ratios (w/w, 1 to 20) in all cell lines. However, the comparison groups such as PEI 25 kDa and Lipofectamine® polyplexes showed below 80% cell viability in all cell lines. In the case of poly(CBA-DAH) polyplexes, there was no toxicity in A549 and Huh-7 cells, whereas the cell viability decreased to 42% in Mai PaCa-2 cells. This result indicates that the poly(CBA-DAH) is seriously toxic to certain cells. The results of MTT assay indicate that PCDP had no cytotoxicity and that it should be safer than comparison groups when it is used to deliver the pDNA.

### Example 5—Cellular Uptake Study

To confirm the cellular uptake of PCDP/pDNA polyplexes, A549, Huh-7, and Mia PaCa-2 cells were seeded at a density of 1×10⁵ cells/well in a 12-well plate in DMEM (10% FBS) and incubated for 24 h before cellular uptake. The medium was replaced by serum-free DMEM, then the cells were treated with polyplexes. The polyplexes of PCDP, poly(CBA-DAH), PEI 25 kDa, and Lipofectamine® were prepared with pDNA (YOYO-1 stained gWiz-Luc, 1 molecule of the dye per 50 bp of nucleotide, 1 μg) using the same protocol as the cytotoxicity experiments. After 4 h, the medium was removed and washed with PBS. The cells were collected by trypsinization and centrifugation (3 min and 3,000 rpm). The degree of cellular uptake was investigated by a BD FAC scan analyzer at a minimum of 1×10⁴ cells gated per sample. The data was analyzed using De NoVo FCS Express 5 Plus software.

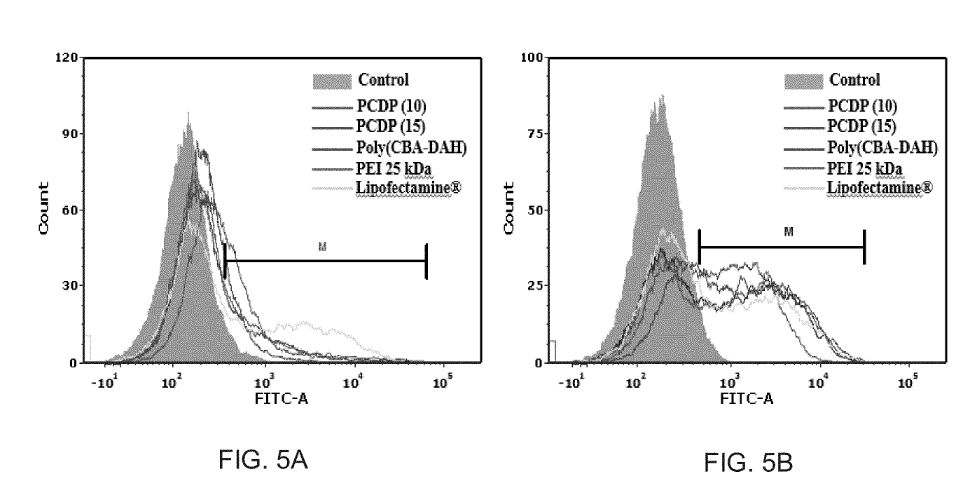

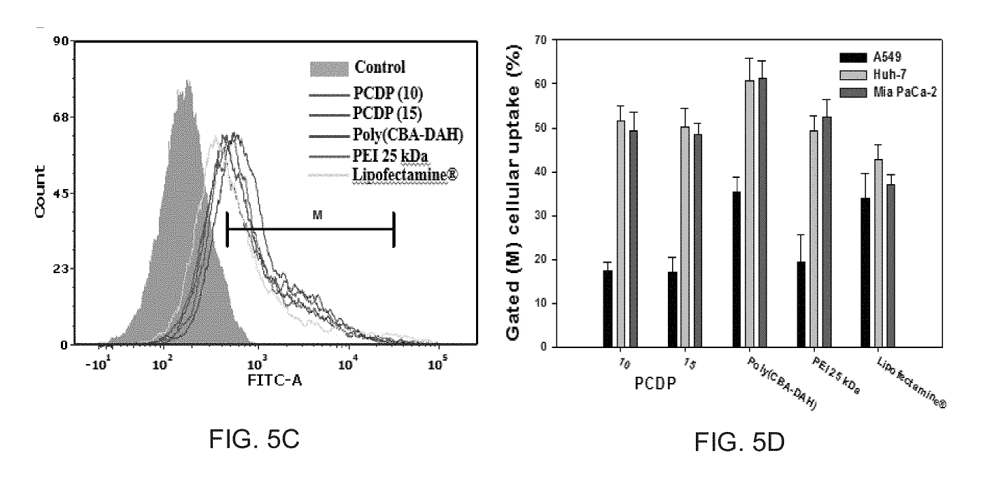

The cellular uptakes of PCDP/pDNA polyplexes were investigated at various weight ratios (1 to 20) by FAC scan analyzer in various cancer cell lines. The polyplexes were formed between polymers and pDNA which was stained by YOYO-1 dye according to the manufacturer's protocol for green fluorescence measurements. Poly(CBA-DAH), PEI 25 kDa, and Lipofectamine® were also used as comparison groups and formed polyplexes at optimized or recommended weight ratio of 40, 1, and 2 based on pDNA (1 μg), respectively. The cellular uptake efficiency of PCPD polyplexes showed low efficiency at weight ratio of 1 and 5, but showed almost similar efficiency at weight ratios of 10 and above in all cell lines (data not shown). To compare with control groups, the cellular uptake efficiency of PCDP polyplexes at weight ratio of 10 and 15 are displayed in FIGS. 5A-5D. As shown in FIGS. 5A-5D, PCDP polyplexes showed similar cellular uptake and quantified cellular uptake (%) with PEI 25 kDa polyplexes in all cell lines and showed better results than Lipofectamine® except that of A549 cells. All tested polymers can form polyplexes with nano-sized particle size and positive surface charge. The particle sizes and zeta potentials were 102-128 nm and 27-34 mV (PCDP), 120 nm and 22 mV (PEI 25 kDa), <200 nm and 40 mV (poly(CBA-DAH)), and 150 nm and 38 mV (Lipofectamine®), respectively. In the case of poly(CBA-DAH) polyplexes, showed highest cellular uptake efficiency in all cell lines. This result was considered to be due to the highest positive charge of poly(CBA-DAH), as well as other factors including shape or surface hydrophobicity. The cellular uptake was affected by not only particle size and positive surface charge but also materials, surface hydrophobicity and shape.

### Example 6—GFP and Luciferase Expression Assay

A549, Huh-7, and Mia PaCa-2 cells were seeded at a density of 5×10⁴ cells/well in a 24-well plate in DMEM (10% FBS) and incubated for 24 h. To confirm the luciferase and GFP, the both gWiz-Luc and gWiz-GFP were used. pDNA (gWiz-Luc or gWiz-GFP) was complexed to form the polyplexes with the PCDP, poly(CBA-DAH), PEI 25 kDa, and Lipofectamine® at various weight ratios described above, respectively. The prepared polyplexes were treated using the same protocol as the cytotoxicity experiments. In addition, to create an in vitro environment that mimics in vivo conditions more accurately, cells were treated with polyplexes (with gWiz-Luc) in 30% FBS.

The GFP expression was visualized after 48 h using an EVOS microscope (AMG, Bothell, Wash.) and each of the GFP expression levels were quantified by measuring the absorbance at 485 nm for excitation and 535 nm for emission on a Tecan Infinite M200 Pro.

For the luciferase analysis, the medium was removed after 48 h, and the cells were washed with DPBS 3 times. Then, the cells were lysed by 1× reporter lysis buffer and collected by scraping and centrifugation. Luciferase quantification was analyzed using a Tecan Infinite M200 Pro (Tecan Group Ltd., Mannedorf, Switzerland). The amount of protein in the cell lysate was measured using a Pierce® BCA protein assay kit (Pierce®, Rockford, Ill.) according to the manufacturer's protocol.

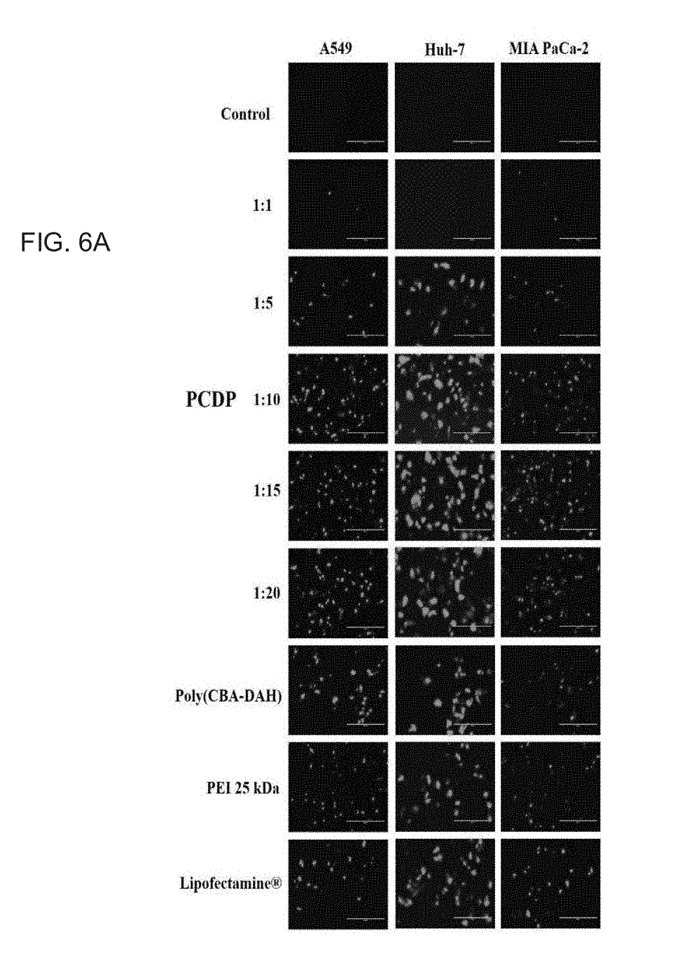

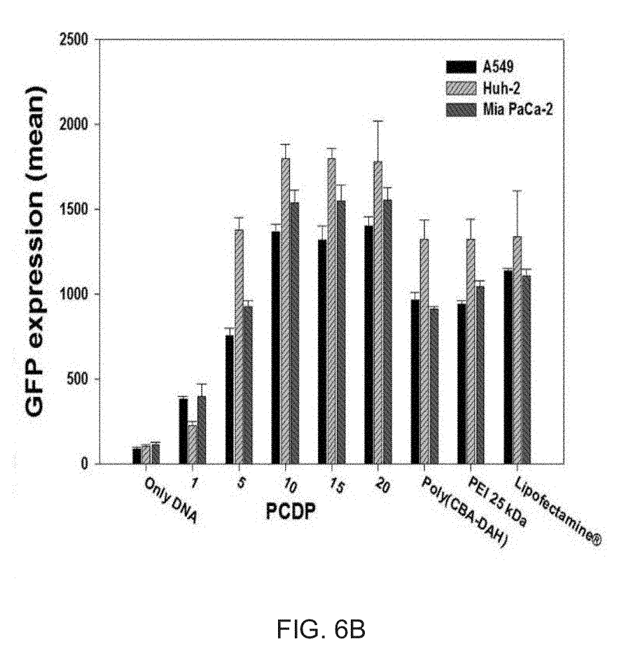

To evaluate the gene transfection efficiency of PCDP polyplexes, both reporter genes gWiz-Luc and gWiz-GFP were used and formed polyplexes with PCDP. The comparison groups including poly(CBA-DAH), PEI 25 kDa, and Lipofectamine® were also treated in various cancer cell lines. The direct visualization of GFP expression of polymer/pDNA (gWiz-GFP) polyplexes are displayed in FIGS. 6A-6B. The GFP gene expression of PCDP polyplexes were increased with increasing polyplexes formation ratios ranging from 1 to 10 (w/w ratio) but showed almost similar results above weight ratio of 10 in all treated cell lines. This result indicates that some optimal ratios are at a weight ratio of 10 or 15. Therefore, the following experiments such as luciferase and VEGF silencing assay of PCDP polyplexes were measured at the optimal weight ratios of 10 and 15. The comparison groups including poly(CBA-DAH), PEI 25 kDa, and Lipofectamine® showed similar GFP gene expression in all cell lines. In the case of PCDP above 10 (w/w ratio), the visualized GFP gene expression and the mean fluorescence intensity (MFI) were higher than other comparison groups. As described above, PCDP has disulfide bonds which can be cleaved by reducing agents such as GSH, allowing pDNA to be easily released from polyplexes into the cytoplasm with no considerable cytotoxicity. Therefore, PCDP polyplexes had highest GFP gene expression efficiency. In addition, poly(CBA-DAH) polyplexes had more cellular uptake than PCDP polyplexes, but PCDP polyplexes showed the increased GFP gene expression efficiency. It may be considered that it can easily escape from the endosome due to increased buffering capacity by PEI conjugation. The MFI of PCDP polyplexes in cell lines followed the rank Huh-7>Mia PaCa-2>A549 cells.

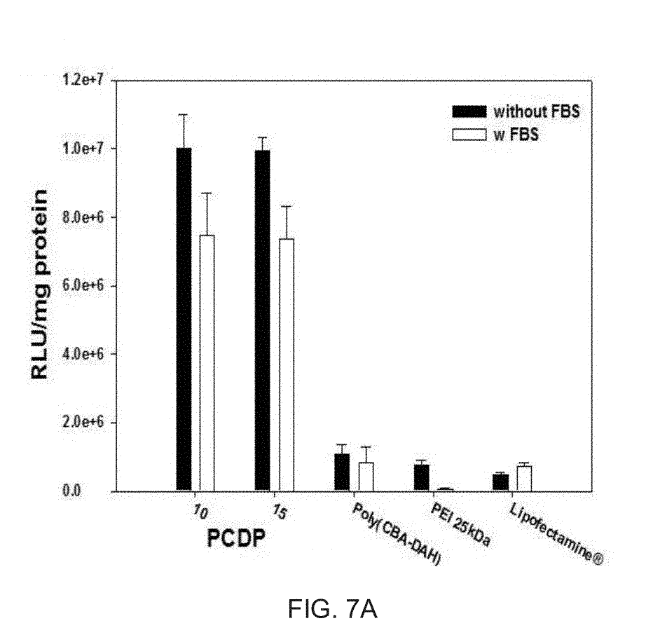

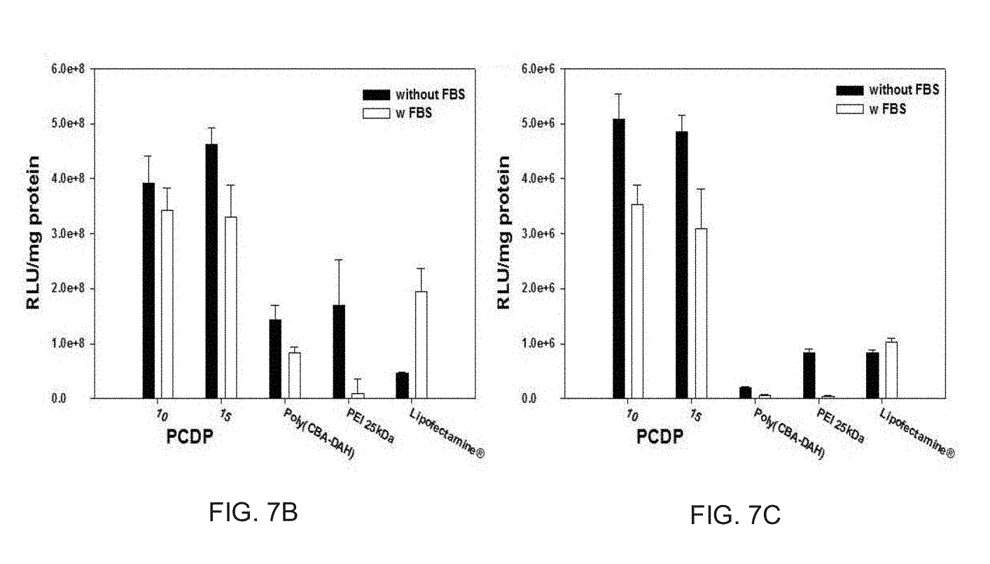

FIGS. 7A-7C show the data of the transfection efficiency using gWiz-Luc. In addition, to mimic in vivo conditions, the transfection efficiency was also evaluated in the presence of FBS (30%). The related light units (RLU) of PCDP at both weight ratio 10 and 15 were higher than other comparison groups in all treated cell lines. These results were consistent with MFI results of the GFP expression assay. The RLU of PCDP, poly(CBA-DAH), and PEI 25 kDa polyplexes was decreased in mimicked in vivo conditions. Interestingly, Lipofectamine® showed increased transfection efficiency at the same conditions. However, the transfection efficiency of PCDP polyplexes was still higher than other comparison groups, even Lipofectamine polyplexes, which had increased transfection efficiency in mimicked in vivo conditions. Especially, PCDP polyplexes showed 10.2 times, 1.7 times, and 6.1 times higher gene transfection efficiency than Lipofectamine® polyplexes in A549, Huh-7, and Mia PaCa-2 cells, respectively. The RLC of PCDP polyplexes in cell lines with or without FBS followed the rank Huh-7>A549>Mia PaCa-2 cells.

### Example 7—VEGF Silencing and Cancer Cell Growth Inhibition of PCDP Polyplexes

VEGF silencing efficacy of PCDP polyplexes was evaluated by ELISA using VEGF siRNA expressing plasmid (pshVEGF). A549, Huh-7, and Mia PaCa-2 cells were seeded at a density of 5×10⁴ cells/well in a 24-well plate, then the PCDP/pshVEGF polyplexes were treated as described above. Poly(CBA-DAH), PEI 25 kDa, and Lipofectamine® were also used as comparison groups. After incubation time (48 h), the medium of each well was harvested and the VEGF expression was measured by human VEGF ELISA kit according to the manufacturer's protocol. In addition, to confirm the cell growth inhibition, the cells were treated with PCDP/pshVEGF polyplexes at weight ratios 10 and 15 as described above and cell viability was investigated by MTT analysis. Cells were also transfected with polyplexes using gWiz-Luc at the same conditions, and the cell viability was investigated by MTT analysis for comparison purposes.

The VEGF siRNA expressing plasmid (pshVEGF) was constructed to inhibit VEGF expression of cancer cells and to express VEGF siRNA longer than directly delivering siRNA. VEGF is a well-known signal protein produced by cells that stimulates vasculogenesis and angiogenesis. The cancer cells can express VEGF, leading to the growth and metastasis of tumors via angiogenesis. Therefore, disabling VEGF receptor function and inhibiting VEGF expression can be used as a strategy to inhibit tumor growth and metastasis.

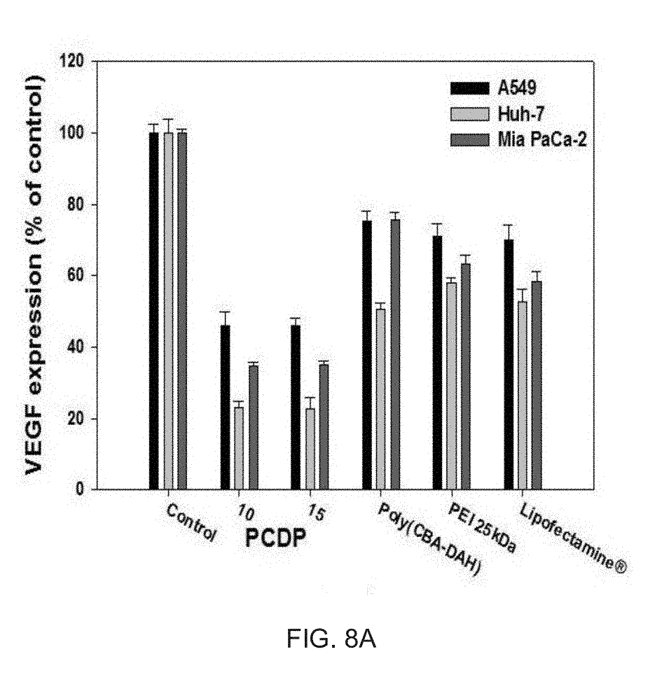

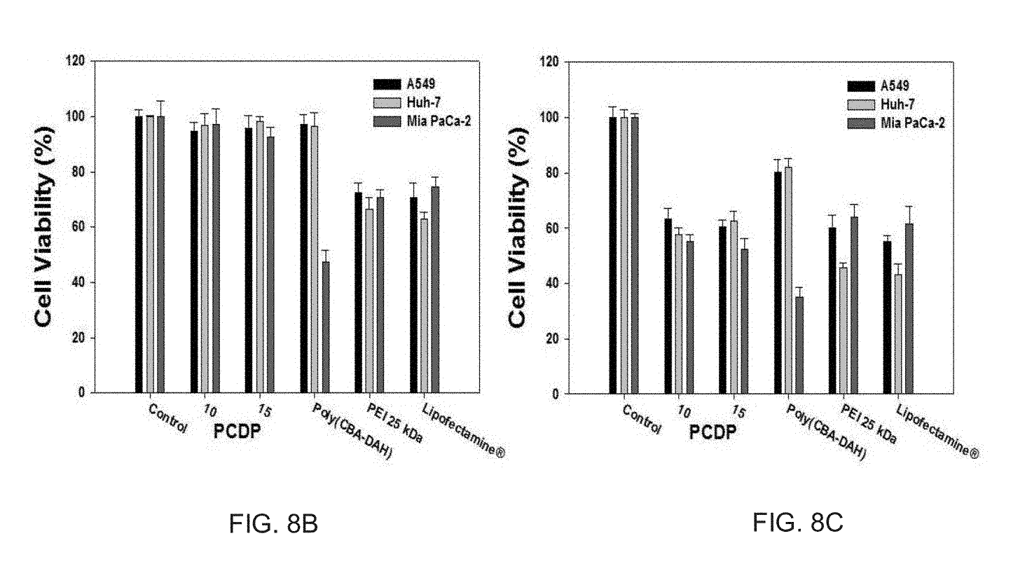

The VEGF gene expressions (% of control) in relation with gene silencing efficiency were measured by human VEGF ELISA in FIG. 8A. The cells were transfected by polymers/pshVEGF polyplexes. The VEGF expressions of PCDP polyplexes at weight ratio of 10 and 15 were dramatically decreased and showed almost similar results between weight ratio of 10 and 15. In addition, the PCDP polyplexes showed lower VEGF expression than poly(CBA-DAH), PEI 25 kDa, and Lipofectamine® polyplexes in all cell lines and these results were consistent with transfection efficiency assay using gWiz-Luc and gWiz-GFP. Compared to non-treated control cells, gene silencing efficiency of PCDP polyplexes showed 54%, 77%, and 66% (w/w; 10) and 55%, 78%, and 65% (w/w; 15) reduction of VEGF levels in A549, Huh-7, and Mia PaCa-2 cells, respectively.

The cellular growth inhibition of PCPD/pshVEGF polyplexes was investigated at the same experimental condition as in ELISA. In addition, to confirm whether or not the inhibition of cell growth was due to VEGF gene silencing or polymer toxicity, the polyplexes using gWiz-Luc was treated into the cells with the same conditions and measured by MTT assay. The cell viabilities of PCDP polyplexes were decreased from 95% (formed with gWiz-Luc) to 60% (formed with pshVEGF) as shown in FIG. 8B and FIG. 8C. This result indicates that the VEGF gene silencing by PCDP/pshVEGF polyplexes indirectly inhibits the cell proliferation and growth rates. The comparison groups, which used pshVEGF, also showed decreased cell viability, even lower than that of PCDP polyplexes. However, the decrease in cell viability is not only from the VEGF siRNA silencing effect but also from the cytotoxicity of the polymers (FIG. 8B). The reduced cell viability rates associated with both results (gWiz and pshVEGF) were 31%, 39%, and 42% (PCDP, w/w; 10), 35%, 36%, and 40% (PCDP, w/w; 15), 17%, 14%, and 12% (poly(CBA-DAH)), 12%, 21%, and 6% (PEI 25 kDa), and 15%, 20%, and 13% (Lipofectamine®) in A549, Huh-7, and Mia PaCa-2 cells, respectively. The PCDP polyplexes with weight ratio of 10 or 15 had similar results in the all experiments. Therefore, the optimal weight ratio (of those tested) of PCDP is 10.

What is claimed is:

1. A bio-reducible polymer, comprising:
poly(ethyleneimine) (PEI) conjugated to poly(cystaminebis(acrylamide)-diaminohexane) (poly(CBA-DAH)).

2. The bio-reducible polymer of claim 1, wherein the PEI has a molecular weight of from about 500 daltons (Da) to about 5 kilodaltons (kDa).

3. The bio-reducible polymer of claim 1, wherein the poly(CBA-DAH) has a general structure according to formula I: 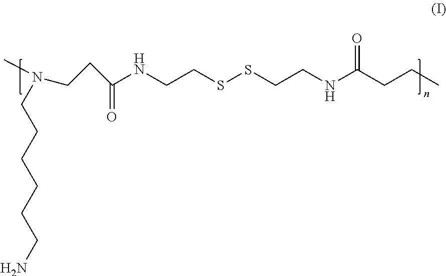
where n is an integer from 1 to 12.

4. The bio-reducible polymer of claim 1, wherein the PEI is conjugated to the poly(CBA-DAH) via a bio-reducible linkage.

5. The bio-reducible polymer of claim 1, wherein the bio-reducible linkage comprises a disulfide bond, an amide bond, an ester bond, an ether bond, or a combination thereof.

6. The bio-reducible polymer of claim 1, wherein the bio-reducible polymer has a general structure according to formula II: 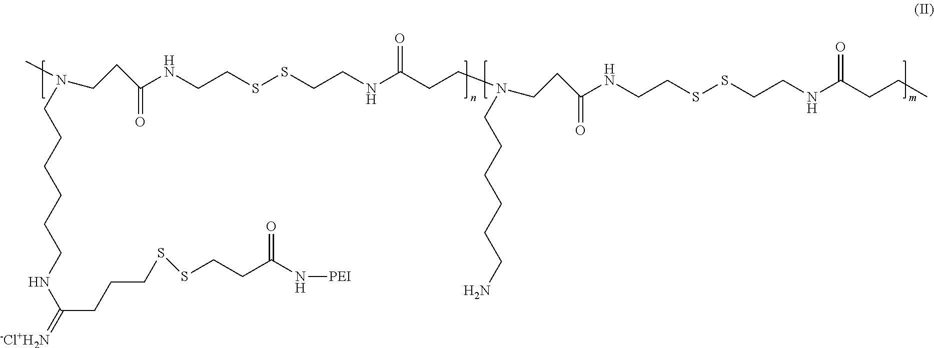
where n is an integer from 1 to 6 and where m is an integer from 1 to 6.

7. The bio-reducible polymer of claim 1, wherein the PEI has a conjugation ratio to poly(CBA-DAH) of from 1 to 8.

8. The bio-reducible polymer of claim 1, wherein the bio-reducible polymer has a buffer capacity that is at least 5% greater than poly(CBA-DAH) alone.

9. A therapeutic agent, comprising:
a therapeutic nucleic acid carried by a bio-reducible polymer of claim 1.

10. The therapeutic agent of claim 9, wherein the therapeutic nucleic acid includes a plasmid deoxyribonucleic acid (pDNA), an anti-sense oligonucleotides (ASO), an aptamer, a ribozyme, a small interfering ribonucleic acid (siRNA), a microRNA (miRNA), a messenger RNA (mRNA), or a combination thereof.

11. The therapeutic agent of claim 9, wherein the bio-reducible polymer and the therapeutic nucleic acid are present at a weight ratio of from 0.5 to 20.

12. The therapeutic agent of claim 9, wherein the therapeutic agent has a particle size of from about 90 nm to about 150 nm.

13. The therapeutic agent of claim 9, wherein the therapeutic agent has a zeta potential of from about 20 mV to about 40 mV.

14. A therapeutic composition, comprising:
a therapeutic agent of claim 9; and
a pharmaceutically acceptable carrier.

15. The therapeutic composition of claim 14, wherein the composition is formulated as an oral dosage form.

16. The therapeutic composition of claim 14, wherein the composition is formulated as an injectable dosage form.

17. The therapeutic composition of claim 14, wherein the composition is formulated as a topical or transdermal dosage form.

18. A method of delivering a therapeutic nucleic acid to a target cell, comprising:
administering a therapeutic agent of claim 9 to the target cell.

19. The method of claim 19, wherein administering the therapeutic agent to the target cell results in modified gene expression of the target cell.

20. The method of claim 19, wherein administering the therapeutic agent has a higher transfection efficiency than an equivalent therapeutic nucleic acid carried by poly(CBA-DAH) alone.
